(19) United States
(12) Patent Application Publication
(10) Pub. No.: US 2009/0007913 A1
(43) Pub. Date: Jan. 8, 2009

(54) Linear motor based respiratory ventilator combining conventional and high frequency ventilation

(76) Inventor: Shouyan Lee, Rancho Santa Margarita, CA (US)

Correspondence Address: WPAT, PC;INTELLECTUAL PROPERTY ATTORNEYS, 2030 MAIN STREET, SUITE 1300, IRVINE, CA, 92614, US

(21) Appl. No.: 12/167,989

(22) Filed: Jul. 3, 2008

Related U.S. Application Data
(63) Continuation of application No. 60/947,936, filed on Jul. 3, 2007.

(51) Int. Cl. A61M 16/00; A62B 9/02; A61B 5/08

(52) U.S. Cl. 128/204.18; 128/205.24; 600/529

(57) ABSTRACT

A linear motor-based respiratory ventilator designed to operate at both high frequency (>1.5 Hz) mode and conventional pressure and volume breath delivery mode, with one or multiple linear motor-gas chamber module controlled by current to the voice coil of the linear motor to eliminate the need for a compressed gas chamber. The ventilator can have a displacement sensor to help determine frequency and then supply data to a processor to control the frequency of the linear motor. In a design where multiple gas chambers are used, blending of gases can be achieved by controlling the displacements of pistons in the multiple gas chambers with the linear motors. A piston can also concurrently control displacement of capacity in more than one gas chamber so that a continuous flow of gas can be achieved.

### CROSS-REFERENCE TO RELATED APPLICATIONS

This application claims priority to U.S. Provisional Pat. No. 60/947,936, filed on Jul. 3, 2007, which is hereby incorporated by reference in its entirety.

Although incorporated by reference in its entirety, no arguments or disclaimers made in the provisional application apply to this non-provisional application. Any disclaimer that may have been stated in the provisional application is hereby expressly rescinded. Consequently, the Patent Office is asked to review the new set of claims in view of all of the prior art of record and any search that the Office deems appropriate.

### BACKGROUND OF THE INVENTION

(1) Field of the Invention

The field of the invention is respiratory ventilator.

(2) Description of Related Art Including Information Disclosed Under 37 CFR 1.97 and 1.98

Various types of respiratory ventilators are known in the medical field. There is, however, a continuing need for new ways to provide high frequency, conventional frequency, continuous bias gas flow, or a mix of these properties in a respiratory ventilator.

All referenced patents, applications and literatures are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply. The invention may seek to satisfy one or more of the above-mentioned desires. Although the present invention may obviate one or more of the above-mentioned desires, it should be understood that some aspects of the invention might not necessarily obviate them.

### BRIEF SUMMARY OF THE INVENTION

Among the many different possibilities contemplated, the contemplated ventilator may utilize linear motors, and specifically linear motors with voice coil actuators. It is further contemplated that a multiple of such linear motor/gas chamber modules are coupled to provided desired properties, such as, blending different gases, multi-waveform, multi-frequency application, high frequency application, continuous flow application.

The displacement of the linear motor is contemplated to be monitored by a displacement sensor, which sends data to a processor to in turn control displacement of the linear motor. The processor receives data from displacement sensor, and from user input (input signal sent from an input device), and in turn controls a power amplifier as the amplifier supplies current to the linear motor. Volume control of the ventilation is controlled by controlling the displacement while the pressure control of the ventilation is controlled by the force of displacement, which directly relates to the current supplied to the voice coil.

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWINGS

FIG. 1 is a schematic view of a first embodiment of a single module in a ventilator system according to an aspect of the inventive subject matter.

FIG. 2 is a schematic view of another embodiment of the ventilator system according to an aspect of the inventive subject matter, where two modules are coupled in a multi-waveform/multi-frequency, high frequency application.

FIG. 3 is a schematic view of another embodiment of the ventilator system according to an aspect of the inventive subject matter, where two modules are coupled in a gas-blending application.

FIG. 4 is a schematic view of another embodiment of the ventilator system according to an aspect of the inventive subject matter, where movement of a piston concurrently affects displacement of two gas chambers in a continuous flow application.

### DETAILED DESCRIPTION OF THE INVENTION

The invention and its various embodiments can now be better understood by turning to the following detailed description of the preferred embodiments, which are presented as illustrated examples of the invention defined in the claims. It is expressly understood that the invention as defined by the claims may be broader than the illustrated embodiments described below. It should also be noted that the drawings are in simplified form and are not to precise scale. In reference to the disclosure herein, for purposes of convenience and clarity only, directional terms, such as, top, bottom, left, right, up, down, over, above, below, beneath, rear, front, distal, and proximal are used with respect to the accompanying drawings. Such directional terms should not be construed to limit the scope of the invention in any manner.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed herein even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims therefore include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

As used herein, the term “gas cylinder” refers generally to a chamber for containing gas to be delivered in a respiratory ventilation system. It should be noted that although the word “cylinder” may be used in this application or in the provisional application to which this application claims priority to, “cylinder” should not be construed to have a specific physical shape. More specifically, contemplated gas chambers in the inventive subject matter are not limited to a cylindrical shape.

As used herein, the term “piston” refers generally to a separator structure, and can be a diaphragm-type separator, used for sealingly provide displacement of a volume of gas inside the gas chamber. It should be noted that the word “piston” should not be construed to have a specific physical shape.

As used herein, the term “mixing” is used interchangeably with “blending.”

FIG. 1 generally depicts the basic schematics of a first embodiment of the contemplated ventilator in accordance with the present invention. Here, a respiratory ventilator system 100 has a first gas chamber 1 having non-compressed gas inside. At rest, the gas inside gas chamber 1 is not in a compressed state. During movement of the piston to deliver gas out of the outflow lumen 2A through outflow check valve 2B, the gas inside the gas chamber 1 is pressurized due to movement of the piston. One key feature of the current invention is to eliminate the need of a compressed gas cylinder.

The outflow lumen 2A is coupled to the first gas cylinder so that gas flowing out of the gas chamber 1 is delivery through the outflow lumen, and to the patient. Outflow check valve 2B is used to regular the direction of gas flow, so that gas only flows in an outward direction in the outflow lumen 2A. The outflow check valve can be positioned in the outflow lumen 2A, or at the entry point of the outflow lumen 2A, or it can be positioned inside of the gas chamber 1.

Preferred ventilator has an inflow opening allowing entry of gas into the gas chamber 1. This inflow opening may optional have an inflow lumen, or it may be a direct opening to the ambient air. Contemplated ventilator has an inflow check valve 3 coupled to the gas chamber 1 to regular direction of gas flow through the inflow check valve 3 such that gas is only allow to enter into the gas chamber 1 through the inflow check valve 3.

Again, the basic module as illustrated in FIG. 1 can be used to deliver breaths without a source of compressed air when valve 3 is open to filtered ambient air. During exhalation, the check valve 2B is closed and check valve 3 is open while the piston is moving towards the right, while filtered ambient air fills the gas chamber 1. During inspiration, valve 2B is open and valve 3 is closed, the gas volume in the chamber is then delivered.

Preferred ventilator can use various known type of actuator to drive a piston 4 to delivery gas form the gas chamber 1 to the patient. Preferably, a linear motor 5A is used to drive the piston 4 that is coupled to the gas chamber 1. Movement of the piston 4 displaces a volume of gas in the gas chamber 1.

Contemplated linear motor 5A is the type having a voice coil 5B disposed within as an actuator.

Voice coil actuators and motors are known to generate force when subjected to an electrical current or magnetic field. Typically, the coil within the motor is the only moving component and is usually attached to a moveable load with a device such as a voice coil valve. This design permits high-speed motion and accurate positioning. Voice coil motors can be an effective alternative to electromechanical components such as servomotors. Voice coil systems do not produce motion with gears or screws, and desirably, nor do they generate heat.

There are two basic types of voice coil products: linear and rotary. In the preferred embodiment, a linear voice coil actuator is used. In less preferred embodiments, a rotary voice coil actuator is used.

Contemplated ventilator 100 has at least one sensor to collect data regarding output and displacement, so as to help adjusting gas output levels. These sensors can include pressure sensors, displacement sensors, gas flow sensors, frequency sensors, and other known sensor for this application. These sensors maybe positioned in various components of the ventilator to collect data. For example, these sensors can be disposed in the gas chamber 1, in outflow lumen 2A, on outflow check valve 2B, on linear motor, on voice coil. These sensors are electronically coupled to a processor, where data are managed and processed into specific commands for adjustment.

In the preferred embodiment, a displacement sensor 6 is used. The contemplated displacement sensor 6 is coupled to at least one of the first linear motor and the first gas chamber. More preferably, the displacement sensor 6 is coupled to the linear motor.

The contemplated respiratory ventilator system can include a user-operable input device 8 so that an operator of the ventilator can enter values desired for each patient. The input device 8 can be a keyboard, a mouse, or other typical data input device known for ventilators. Input signal X from the input device are sent to the processor 9, wherein the processor 9 receives the input signal X and a displacement signal Y from the first displacement sensor.

Preferred respiratory ventilator system can have a power amplifier 7 electronically coupled to the linear motor 5A, wherein the power amplifier receives a command Z from the processor 9, and the power amplifier 7 supplies a current C to said first linear motor 5A, and wherein the current C is variably and adjustably controlled by the processor 9. Current C is preferably DC current.

Operation of the ventilator is straightforward. An operator enters desired value of ventilation using input device 8, input signal X is sent from the input device 8 to processor 9. Processor 9 sends command signal Z to power amplifier 7, so that power amplifier is activated to send current C to linear motor 5A. Current C drives voice coil 5B in a linear motion. Because voice coil 5B is coupled to piston 4, piston 4 also moves in a linear motion within the gas chamber 1. Because piston 4 making sealing contact with the walls of the gas chamber 1, movement of piston 4 effectively changes the volume capacity of the gas chamber 1. Referring to FIG. 1 as an illustration, when piston 4 moves toward left, volume capacity of gas chamber 1 decreases, forcing a volume of gas to exit gas chamber through outflow check valve 2B, and through outflow lumen 2A to the patient. The processor periodically, or constantly, processes data collected from displacement sensor 6 to help determine the adjustment needed to keep total output within the desired value as entered by the operator. In general, volume control of the ventilation is controlled by varying the displacement range of the piston, and pressure control of the ventilation is controlled by varying the thrusting force of the piston which can be controlled by varying the current supplied to the voice coil.

In an application to provide conventional ventilation under Volume Mode, volume and flow is servo controlled by the displacement so that Volume=Displacement*Area of the piston and Flow=dV/dt.

In an application to provide conventional ventilation under Pressure Mode, pressure is directly controlled by the current to the voice coil, since Pressure*Area of the Piston=Force=BLI, where B is the strength of the magnetic field, L is the length of the coil and I the current. Contemplated system can be fine-tuned with a downstream pressure transducer.

FIG. 1 and the description above generally illustrate the embodiment of contemplated linear motor-based respiratory ventilation system, or a single module that can be used in a multiple module respiratory ventilation system.

In situations where higher frequency of ventilation is desired, in a single module system such as that shown in FIG. 1, the voice coil 5 can increase frequency of movement to achieve the desired high frequency.

In another contemplated embodiment, high frequency output can be achieved by having multiple modules in a ventilation system.

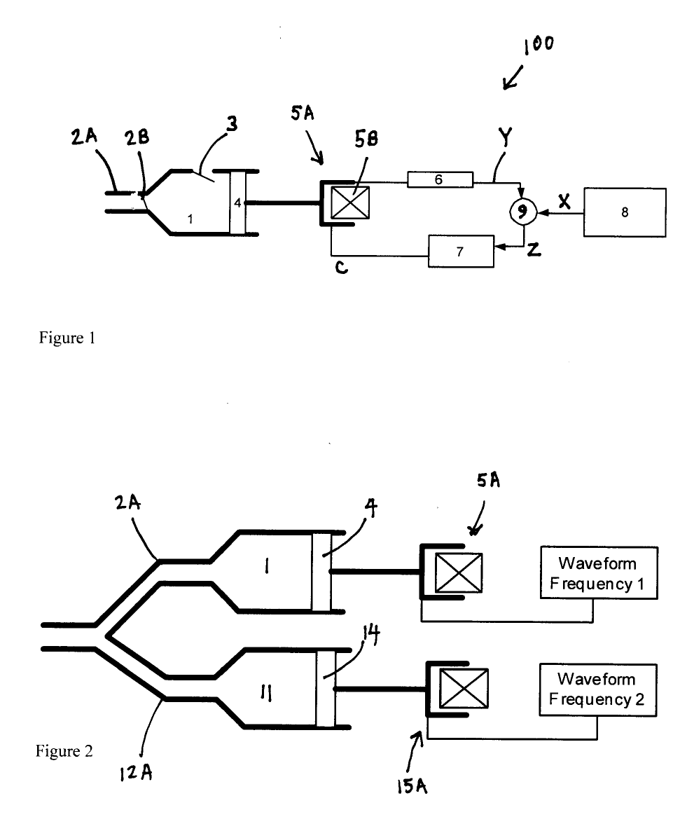

Referring now to FIG. 2, the contemplated system can have a high frequency mode to produce multiple frequency. Here, by using system with multiple basic modules, the system can deliver high frequency breaths with multiple frequencies and waveforms.

The contemplated respiratory ventilator system can further have a second gas chamber 11 having a second outflow lumen 12A and a second outflow check valve 12B. The second outflow lumen 12A is fluidly coupled to the first outflow lumen 2A so that eventually a single source of gas ventilation to supplied to the patient. Gas chamber 11 has a second piston 14 sealingly and movably coupled to the second gas chamber 11 to control a volume of capacity in the second gas chamber 11. A second volume of gas is disposed in the second gas chamber 11. A second linear motor 15A is coupled to the piston 14 to drive the piston 14 to move in a linear motion similar to the first module. Essentially, multiple modules are coupled together to produce a total output of ventilation.

In one embodiment, the first linear motor 5A creates a first waveform and a first frequency, and wherein the second linear motor 15A creates a second waveform and a second frequency. The first frequency is different from the second frequency. In another embodiment, the two frequencies are substantially the same, and both provide outflow of gas at the same time so as to produce a desired volume of output.

In a preferred embodiment, the two frequencies are the same, but the timing of output for both modules are staggered, or equally spaced apart, such that when the first module produce an output, module 2 does not. In essence, the waveforms of the two modules are different in that their peaks are spaced apart. This staggered output can produce a higher total output frequency than a frequency that can be achieved by a single module alone.

In a preferred embodiment, the contemplated multi-module respiratory ventilator system can have a total frequency output of more than 1.0 Hz. More preferably, the total frequency output is more than 1.5 Hz. Even more preferably, the total frequency output is more than 1.7 Hz.

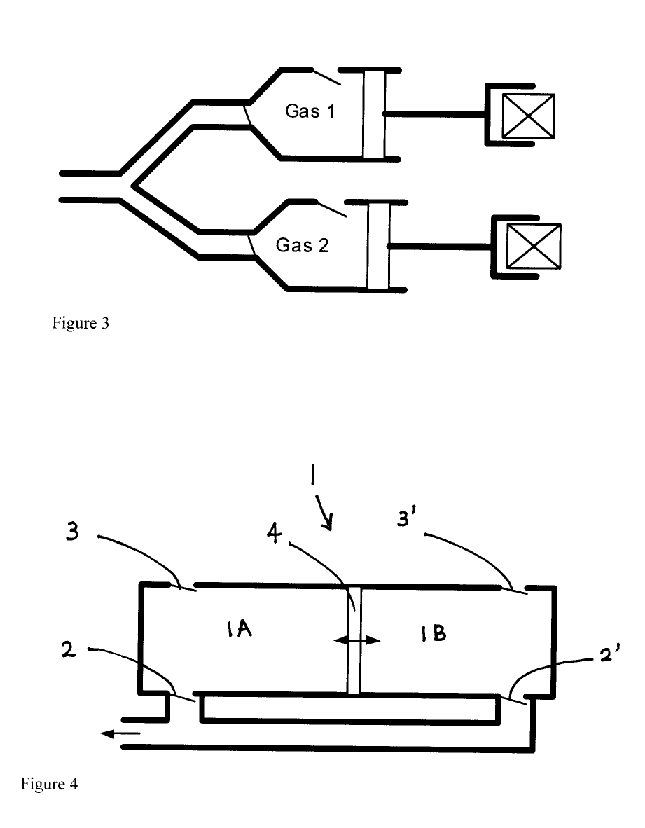

Referring now to FIG. 3. Another contemplated advantage of a multi-module system is the ability to blend gases. In a contemplated system, the first volume of gas can be a different gas from the second volume of gas. One or some of the modules in the system can work independently from other modules, and can be controlled separately from other modules. For example, delivery of certain gases can be on standby, until given the signal by the processor 9 to start delivery. Outflow lumens of multiple modules are fluidly coupled. For example, in FIG. 3, coupling of first and second outflow lumens allows mixing of the first and second volume of gases from the two different modules.

In other embodiments, accurate gas blending can be achieved by controlling two or more basic modules with different volumes for each gas components preset to obtain the desired gas mixture.

Now referring to FIG. 4, an embodiment of system to provide continuous bias flow without a source of compressed air is provided.

FIG. 4 shows a modification of the basic module. It can be described as having a single gas chamber divided by a diaphragm-type piston, or it can be described as two gas chambers coupled together jointly using a single diaphragm-type piston.

With two more check valves 2′ and 3′ added to the gas chamber 1, both side of the diaphragm-type piston 4 can be used to deliver a continuous bias air flow. The diaphragm sealingly and movably divides the gas chamber 1 into chamber 1A and chamber 1B. When the piston 4 moves towards the right, valve 2′ opens to deliver airflow from chamber 1B (and valve 3′ is closed) while valve 3 opens to fill the chamber 1A (and valve 2 is closed). When the motion of the piston 4 is reversed, valve 2 opens to deliver the airflow from chamber 1A and valve 3′ opens to fill chamber 1B.

In other words, chamber 1B has a second outflow lumen fluidly coupled to the first outflow lumen, wherein displacement of the first piston 4 also changes a capacity of volume in the second gas chamber 1B, wherein a single movement of the piston 4 decreases a first capacity of the first gas chamber 1A, forcing the first volume of gas inside to exit the first gas chamber 1A, while the same single movement of the first piston increases a second capacity of the second gas chamber, forcing a second volume of gas to enter into the second gas chamber 1B.

A complete cycle of piston will have continuous airflow delivery. Two or more linear motor modules as shown in FIG. 4 may be added to switch the direction of piston motion at different time of the cycle, to minimize the flow transient and motion interruption when the piston switches direction, and the check valves open and close.

Preferred ventilator system may comprise as many basic modules as necessary to achieve combined functions such as gas blending, multi-frequency mode, continuous bias flow etc. Preferred ventilator system may also mix and match basic modules are illustrated in FIG. 1, with continuous flow modules as shown in FIG. 4.

As those of ordinary skill in the art will recognize, the shape, material, and size of the various components in the contemplated ventilation system may readily be modified as dictated by the aesthetic or functional needs of particular applications.

Thus, specific embodiments and applications of linear motor-based respiratory ventilator have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Insubstantial changes from the claimed subject matter as viewed by a person with ordinary skill in the art, now known or later devised, are expressly contemplated as being equivalent within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be obviously substituted and also what essentially incorporates the essential idea of the invention. In addition, where the specification and claims refer to at least one of something selected from the group consisting of A, B, C . . . and N, the text should be interpreted as requiring only one element from the group, not A plus N, or B plus N, etc.

What is claimed is:

1. A respiratory ventilator system, said ventilator system comprises:
a first gas chamber having non-compressed gas;
an first outflow lumen coupled to said first gas cylinder;
an first inflow check valve coupled to said first gas chamber;
a first linear motor; and
a first piston coupled to said first gas chamber and driven by said first liner motor to displace a first volume of gas in said first gas chamber.

2. The respiratory ventilator system of claim 1 further comprising a first outflow check valve disposed in the first outflow lumen

3. The respiratory ventilator system of claim 2 further comprising a first displacement sensor coupled to at least one of the first linear motor and the first gas chamber.

4. The respiratory ventilator system of claim 3 further comprising a first voice coil disposed in the first linear motor.

5. The respiratory ventilator system of claim 4 further comprising a first input device that sends input signal to a processor, wherein the processor receives the input signal and a displacement signal from the first displacement sensor.

6. The respiratory ventilator system of claim 5 further comprising a power amplifier electronically coupled to the first linear motor, wherein the power amplifier receives a command from the processor, and the power amplifier supplies a current to said first linear motor, and wherein the current is variably and adjustably controlled by the processor.

7. The respiratory ventilator system of claim 6 further comprising a second gas chamber having a second outflow lumen fluidly coupled to the first outflow lumen, a second piston coupled to the second gas chamber to displace a second volume of gas in the second gas chamber, a second linear motor coupled to the piston to drive the piston to move in a linear motion, and wherein the first linear motor creates a first waveform and a first frequency, and wherein the second linear motor creates a second waveform and a second frequency.

7. The respiratory ventilator system of claim 7, wherein the first frequency is different from the second frequency.

8. The respiratory ventilator system of claim 7, wherein a total frequency output of the ventilator system is more than 1.5 Hz.

10. The respiratory ventilator system of claim 7, wherein the first volume of gas is a different gas from the second volume of gas, and the fluidly coupled first and second outflow lumens allow mixing of the first and second volume of gases.

11. The respiratory ventilator system of claim 6 further comprising a second gas chamber having a second outflow lumen fluidly coupled to the first outflow lumen, wherein displacement of the first piston also changes a capacity of volume in the second gas chamber.

12. The respiratory ventilator system of claim 11, wherein a single movement of the first piston decreases a first capacity of the first gas chamber, forcing the first volume of gas inside to exit the first gas chamber, while the same single movement of the first piston increases a second capacity of the second gas chamber, forcing a second volume of gas to enter into the second gas chamber.

13. A linear motor-based respiratory ventilator system comprising:
at least one gas delivery module, wherein each module comprises a gas chamber, a piston coupled to the gas chamber, a linear motor having a voice coil that drives the piston to move in a linear motion, and wherein the actuation of the linear motor is controlled by a processor.

14. The ventilator system of claim 13, having at least two gas delivery modules, each module having at least one outflow lumen, and wherein at least one outflow lumen from a gas delivery module are fluidly coupled to the outflow lumen of another module.

15. The ventilator system of claim 14, wherein each of at least two gas delivery modules delivers gas at its own frequency and waveform, and effectuating a high frequency total output of gas to a patient of at least 1.5 Hz.

16. The ventilator system of claim 15 further comprising at least one displacement sensor coupled to at least one linear motor, wherein the at least one sensor sends displacement data to the processor.

17. The ventilator system of claim 16, wherein at least one of the gas delivery modules is a continuous flow module, wherein the continuous flow module has a first gas chamber coupled to a second gas chamber having a second outflow lumen, and wherein a single movement of the piston decreases a first capacity of the first gas chamber, forcing the first volume of gas inside to exit the first gas chamber, while the same single movement of the piston increases a second capacity of the second gas chamber, forcing a second volume of gas to enter into the second gas chamber.

18. The ventilator system of claim 17, wherein when the piston moves in reverse direction from the first single movement, the piston increases the first capacity of the first gas chamber, forcing the first volume of gas to enter the first gas chamber, while at substantially the same time the piston decreases the second capacity of the second gas chamber, forcing the second volume of gas to exit through the second outflow lumen.
